(19) United States
(12) Patent Application Publication
(10) Pub. No.: US 2024/0398226 A1
(43) Pub. Date: Dec. 5, 2024

(54) ATTACHABLE/DETACHABLE STRABISMUS MEASUREMENT DEVICE

(71) Applicant: TI INC., Seoul (KR)

(72) Inventors: Hong Jai LEE, Seoul (KR); Hyeon Wu JEON, Goyang-si (KR)

(21) Appl. No.: 18/799,947

(22) Filed: Aug. 9, 2024

Related U.S. Application Data
(63) Continuation of application No. PCT/KR2022/002144, filed on Feb. 14, 2022.

(30) Foreign Application Priority Data
Feb. 11, 2022 (KR) 10-2022-0018196

(51) Int. Cl. A61B 3/113; A61B 3/00

(52) U.S. Cl. CPC A61B 3/113; A61B 3/0016

(57) ABSTRACT

Provided relates to a device for measuring ocular strabismus. More specifically, the ocular strabismus measuring device according to the present invention includes a covering part, a covering control device, and a coupling part. The device allows for the alternate cover test by selectively covering a variable translucent film controlled by the covering control device. It can be attached to and detached from an eye-tracking device, enabling alternate covering for each eye individually. The device transmits data on the covering state along with binocular eye deviation data to the eye-tracking device. The covering part is easily attachable and detachable, making the device useful for measuring strabismus in infants and children, and it allows for continuous use by replacing the covering part in case of damage or wear.

### TECHNICAL FIELD

The present invention relates to a device for measuring strabismus of the eye, which includes a covering part, a covering control device, and a coupling part.

### BACKGROUND ART

Our eyes normally exhibit orthotropia, where both eyes are always directed towards the object being viewed.

Strabismus is an ocular motor disorder where the two eyes point in different directions.

Strabismus can be classified into various types, primarily divided into
esotropia, where the eyes turn inward, and exotropia, where the eyes turn outward. Esotropia includes “infantile esotropia” and “accommodative esotropia.” Infantile esotropia occurs within the first six months of life. Accommodative esotropia, which results from significant hyperopia (farsightedness), typically appears around the age of 2-3 years. In exotropia, the most common type in Korea is intermittent exotropia, where the eyes align when focusing but deviate when looking at distant objects or when staring blankly. This type is often characterized by intermittent occurrence when tired or not concentrating, leading to delayed detection and missed treatment opportunities. If left untreated, it can develop into “constant exotropia,” where the eyes are always turned outward, thus requiring careful monitoring.

If a child develops strabismus, their vision may not develop properly, leading to poor corrected vision. The longer it takes one eye to turn, the greater the risk of amblyopia (a condition where vision is poor even with glasses). In the case of strabismus, the images formed on the retina of each eye differ, resulting in a lack of depth perception. Symptoms include the child's eyes turning outward when tired or staring blankly, frequent blinking, and rubbing of the eyes. If the frequency and duration of the eye turning increase, or if the child frequently squints due to light sensitivity, strabismus should be suspected.

There are three main diagnostic methods for strabismus: the “Cover-Uncover Test”, the “Prism Alternate Cover Test”, and the “Hirschberg Test”. The Cover-Uncover Test involves covering one eye while the uncovered eye focuses on an object, then removing the cover to observe any movement in the previously covered eye, indicating strabismus. The Prism Alternate Cover Test uses prisms of varying powers placed in front of the eyes; the eyes are alternately covered and uncovered to find the prism power that results in the least movement of the eye, indicating the degree of strabismus. The Hirschberg Test involves shining a light into both eyes and observing the position of the light reflection on the corneas to diagnose strabismus.

Recently, VR devices and “strabismus angle measurement devices” that analyze eye movements have also been developed for diagnosing strabismus.

The Cover-Uncover Test involves having the patient focus on a fixation point with both eyes, then covering the left eye to observe the movement of the right eye and covering the right eye to observe the movement of the left eye. The Alternate Cover Test also starts with the patient focusing on a fixation point with both eyes. Then, the right eye is covered and uncovered quickly while immediately covering the left eye, observing the movement of the previously covered right eye. This process is repeated by alternately covering each eye and observing their movements. The Prism Cover Test is performed during the Alternate Cover Test by placing a prism in front of one eye to measure the degree of eye movement.

The commonly used test for diagnosing strabismus is the Alternate Cover Test, which determines the presence of strabismus and measures the strabismus angle using prisms. During the Alternate Cover Test, the examiner judges the eye movements while alternately covering each eye, and the strabismus angle is measured based on these observations. Traditional methods of the Alternate Cover Test and the Prism Cover Test require the physician to manually perform the test repeatedly, which can be cumbersome and lead to errors due to variations in the duration of covering and the distance of eye movements assessed by different examiners. Recently, VR devices and “strabismus angle measurement devices” that analyze eye movements have been developed. However, existing testing devices have limitations: they lack a mechanism for alternate covering to detect latent strabismus by preventing the fusion of both eyes, and they do not provide information on which eye is fixating, making it difficult to accurately reflect this in the diagnosis.

Accordingly, there is a need for a strabismus measuring device that can be attached to an eye-tracking device, enabling alternate covering of each eye and capable of digitizing and transmitting the eye deviation observed when one eye is covered.

In response, the inventors have developed a strabismus measuring device that includes a covering part, a covering control device, and a coupling part, which can be attached to and detached from an eye-tracking device. The covering control device allows a variable translucent film to switch between a transparent state, providing clear vision, and an opaque state, blocking vision, for each eye individually, enabling the Alternate Cover Test. The device can transmit data on the covering state along with binocular eye deviation data measured by the eye-tracking device. The covering part is easily attachable and detachable, making the device useful for measuring strabismus in infants and children. Additionally, the covering part can be replaced if damaged or worn, allowing for continuous use. This invention thus fulfills the need for an effective strabismus measuring device.

### DESCRIPTION OF THE INVENTION

### Objective to be Solved

The present invention aims to solve the problems by providing a strabismus measuring device that includes a covering part, a covering control device, and a coupling part. This device can be attached to and detached from an eye-tracking device. The covering control device allows a variable translucent film to switch between a transparent state, providing clear vision, and an opaque state, blocking vision, for each eye individually, enabling the Alternate Cover Test. The device can transmit data on the covering state along with binocular eye deviation data measured by the eye-tracking device. The covering part is easily attachable and detachable, making the device useful for measuring strabismus in infants and children. Additionally, the covering part can be replaced if damaged or worn, allowing for continuous use. The objective of this invention is to provide a strabismus measuring device that addresses these needs.

### Solution to the Problem

To solve the problem, the present invention provides a strabismus measuring device that includes a covering part, a covering control device, and a coupling part. This device can be attached to and detached from an eye-tracking device. The covering control device allows a variable translucent film to switch between a transparent state, which provides clear vision, and an opaque state, which blocks vision, for each eye individually, enabling the Alternate Cover Test. The device can transmit data on the covering state along with binocular eye deviation data measured by the eye-tracking device. The covering part is easily attachable and detachable, making the device useful for measuring strabismus in infants and children. Additionally, the covering part can be replaced if damaged or worn, allowing for continuous use. This invention thus provides a strabismus measuring device that addresses these needs.

### Effect of the Invention

The strabismus measuring device of the present invention can be attached to and detached from an eye-tracking device. The covering control device enables a variable translucent film to switch between a transparent state, allowing clear vision, and an opaque state, blocking vision, for each eye individually, making the Alternate Cover Test possible. The device can transmit data on the covering state along with binocular eye deviation data measured by the eye-tracking device. The covering part is easily attachable and detachable, making the device useful for measuring strabismus in infants and children. Additionally, the covering part can be replaced if damaged or worn, allowing for continuous use.

### BRIEF DESCRIPTION OF THE DRAWINGS

FIG. 1 illustrates the strabismus measuring device according to the present invention.

FIG. 2A is a diagram explaining the components of the strabismus measuring device according to the present invention.

FIG. 2B shows an example of the strabismus measuring device according to the present invention.

FIG. 3A is a diagram explaining the configuration of the covering part in the strabismus measuring device according to the present invention.

FIG. 3B shows an example of the covering part in the strabismus measuring device according to the present invention.

FIG. 4 is a diagram explaining the configuration of the covering control device in the strabismus measuring device according to the present invention.

FIG. 5A is a diagram explaining the configuration of the coupling part in the strabismus measuring device according to the present invention.

FIG. 5B shows an example of the coupling part in the strabismus measuring device according to the present invention.

FIG. 6 illustrates the sequence of the alternate cover method according to one embodiment of the strabismus measuring device of the present invention.

### DETAILED DESCRIPTION FOR IMPLEMENTING THE INVENTION

The language used in this specification and claims should not be limited to the ordinary or dictionary meanings but should be interpreted in light of the inventor's intention to explain the invention in the best possible way. The terms should be construed according to the meaning and concept consistent with the technical spirit of the invention, based on the principle that the inventor can appropriately define the concepts of terms. Therefore, the structures described in the embodiments of this specification represent only the most preferred embodiment of the present invention and do not represent all of the technical spirit of the invention. It should be understood that various equivalents and modifications can be made at the time of this application.

Hereinafter, the present invention will be described in detail with reference to the drawings.

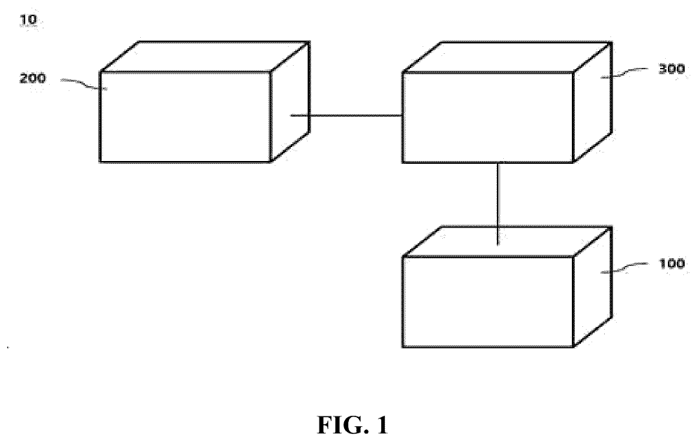

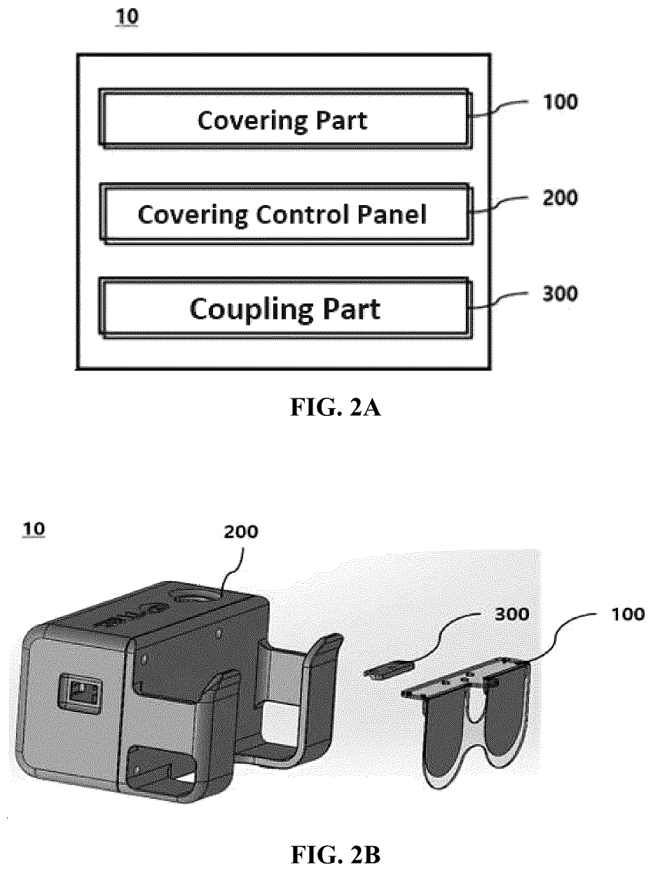

FIGS. 1 and 2A-2B are diagrams explaining an example of the strabismus measuring device according to the present invention.

Referring to FIGS. 1 and 2A-2B, the strabismus measuring device 10 according to one embodiment of the present invention includes a covering part 100, a covering control device 200, and a coupling part 300. The strabismus measuring device, which can be attached and detached, includes an optometry frame mounted on the eye-tracking device, and the coupling part, covering part, and covering control device combined with the optometry frame. The coupling part and covering part can be attached and detached using magnets.

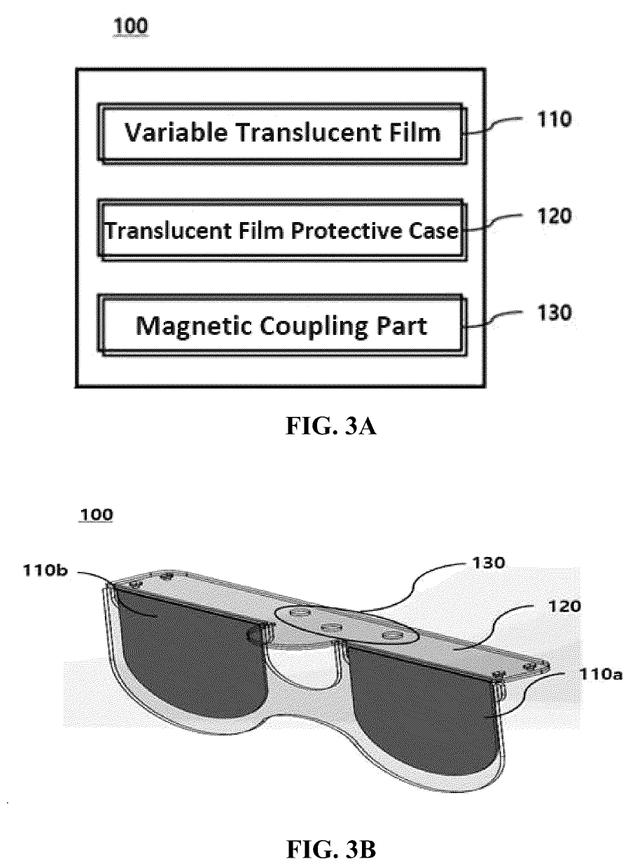

FIGS. 3A-3B are diagrams explaining an example of the covering part 100 in the strabismus measuring device according to the present invention.

As shown in FIGS. 3A-3B, the covering part 100 in the strabismus measuring device includes a variable translucent film 110, a translucent film protective case 120, and a magnetic coupling part 130. The electrode of the coupling part is coupled by the magnetic force of the magnet included in the magnetic coupling part. The covering part 100 is easily attachable and detachable due to the magnetic coupling, allowing for continuous use of the device by replacing the covering part in case of damage or wear. The variable translucent film 110 can be a film or lens but is not limited to these. The film becomes transparent to allow vision when controlled by the covering control device, and becomes opaque to block vision, enabling the alternate cover test by selective covering. The magnet included in the magnetic coupling part 130 is conductive, allowing current to flow through the variable translucent film 110 when power is supplied from the covering control device 200 to the coupling part 300. The magnet can be a neodymium magnet but is not limited to this.

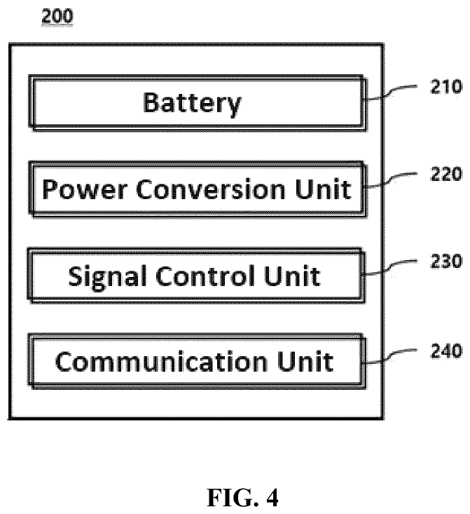

FIG. 4 explains an example of the covering control device in the strabismus measuring device according to the present invention.

As shown in FIG. 4, the covering control device 200 includes a battery 210, a power conversion unit 220, a signal control unit 230, and a communication unit 240. The battery 210 includes a charging terminal for recharging when discharged and supplies power to the power conversion unit 220, the signal control unit 230, and the communication unit 240. The power conversion unit 220 converts the power supplied from the battery 210 to the power used by the variable translucent film. The signal control unit 230 operates by receiving power from the battery 210 and controls the state of the variable translucent film in the covering part 100 to either block or allow vision in a binocular, monocular, or alternating cover test state, and digitizes the real-time state of the covering part. The communication unit 240 transmits the digitized alternating cover state in real time.

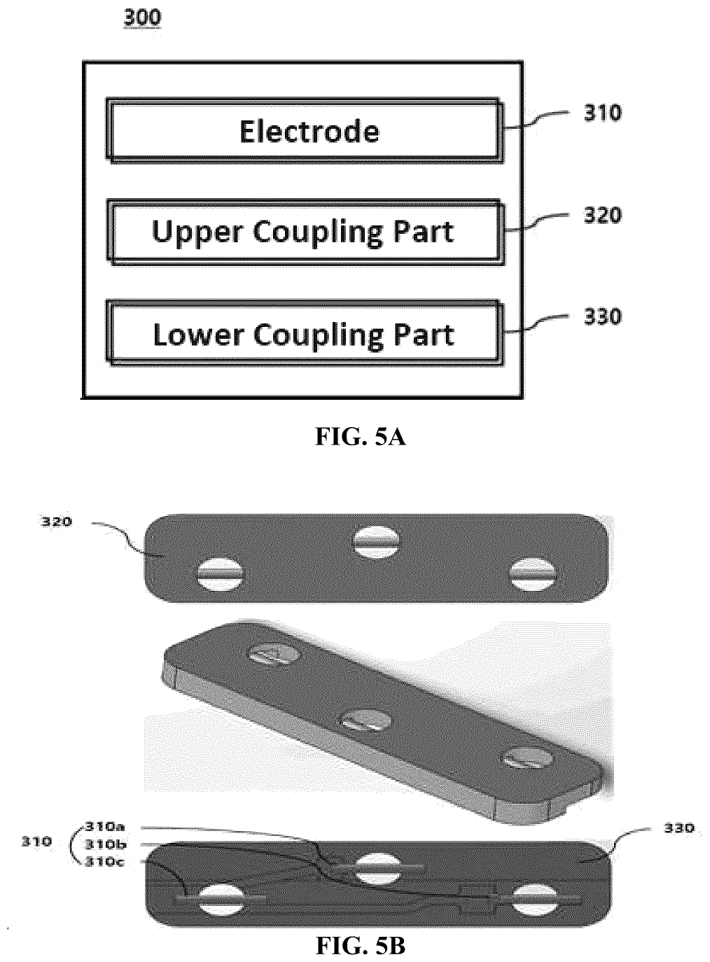

FIGS. 5A-5B explain examples of the coupling part in the strabismus measuring device according to the present invention.

As shown in FIGS. 5A-5B, the coupling part 300 includes an electrode 310 through which current flows, an upper coupling part 320 that couples with the magnetic coupling part 130 of the covering part 100, and a lower coupling part 330 that couples with the eye-tracking device. The eye-tracking device for measuring strabismus can output images for both eyes to focus on, allowing the examiner to measure eye deviation by identifying the exact position of the object being viewed, whether near or far. The images output by the eye-tracking device can be varied according to the purpose of strabismus measurement.

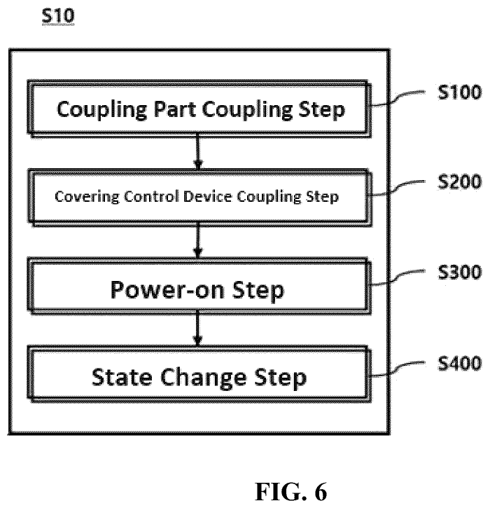

FIG. 6 illustrates the sequence of the alternate cover method according to one embodiment of the strabismus measuring device of the present invention.

As shown in FIG. 6, when the covering control device 200 supplies power, it is transmitted through the electrodes 310 of the coupling part 300 and the magnets in the covering part 100 to the variable translucent film 110. The device can selectively control the states of binocular blocking, binocular vision, and alternating cover test states. The alternating cover device sequence (S10) includes a coupling part coupling step (S100), a covering control device coupling step (S200), a covering part coupling step (S300), a power-on step (S400), and a state change step (S500). In the coupling part coupling step (S100), the covering part 100 is attached to the eye-tracking device.

In the covering control device coupling step (S200), the coupling part 300 and the covering control device 200 are connected by a wire. In the covering part coupling step (S300), the coupling part 300 and the covering part 100 are coupled by the magnetic force of the magnet. In the power-on step (S400), the power of the covering control device 200 is turned on, supplying power through the wires to the magnet in the covering part 100, changing the variable translucent film 110 from opaque to transparent. In the state change step (S500), the user switches the covering state by pressing a button on the covering control device 200, controlling the covering state of the variable translucent film to switch between binocular vision, binocular blocking, and alternating cover test states according to the desired protocol. The covering control device 200 transmits the digitized real-time alternating cover state data wirelessly or through a wired connection.

So far, the present invention has been described with a focus on its preferred embodiments. Those skilled in the art will understand that the invention can be implemented in modified forms without departing from its essential characteristics. Therefore, the disclosed embodiments should be considered in a descriptive sense rather than a restrictive sense. The scope of the present invention is indicated by the appended claims, and all differences within the equivalent scope should be construed as being included in the present invention.

### EXPLANATION OF SYMBOLS

10: Strabismus measuring device
100: Covering part
110: Variable translucent film
120: Translucent film protective case
130: Magnetic coupling part
200: Covering control device
210: Battery
220: Power conversion unit
230: Signal control unit
240: Communication unit
300: Coupling part
310: Electrode through which current flows
320: Upper coupling part
330: Lower coupling part

1. A detachable strabismus measuring device comprising: a coupling part; a covering part;
and a covering control device, wherein the coupling part and the covering part are detachable.

2. The strabismus measuring device of claim 1, wherein the coupling part and the covering part are detachable through magnetic coupling.

3. The strabismus measuring device of claim 1, wherein the device is detachable using the electrode of the coupling part and the magnet of the covering part.

4. The strabismus measuring device of claim 1, wherein the covering part includes a variable translucent film that becomes transparent when current is supplied from the covering control device.

5. The strabismus measuring device of claim 2, wherein the magnet of the covering part is coated with a conductive material allowing current to flow, making it detachable.

6. The strabismus measuring device of claim 2, wherein the magnet of the covering part couples with the electrode of the coupling part to supply current to the variable translucent film.

7. The strabismus measuring device of claim 6, wherein the variable translucent film of the covering part switches between transparent and opaque states as current flows from the electrode of the coupling part through the magnet to the variable translucent film, controlled by the covering control device.

8. The strabismus measuring device of claim 1, wherein the covering control device adjusts the state of the variable translucent film to transparent or opaque, controlling the eyes in a non-covering state, covering state, or alternating cover state.

9. The strabismus measuring device of claim 1, wherein the covering control device controls the covering state of the variable translucent film and transmits the digitized covering state wirelessly or through a wired connection.

10. The strabismus measuring device of claim 9, wherein the digitized covering state is synchronized with eye deviation data.

11. The strabismus measuring device of claim 10, wherein the synchronized data allows verification of both the alternating cover state and eye deviation.

12. A method for measuring strabismus comprising: attaching the coupling part of the strabismus measuring device to an eye-tracking device; connecting the coupling part and the covering control device with a wire; coupling the coupling part and the covering part through magnetic force; supplying power through the wire from the covering control device, changing the variable translucent film from opaque to transparent; switching the covering state by pressing a button on the covering control device, controlling the covering state of the variable translucent film to select a non-covering state, covering state, or alternating cover state, or according to a desired protocol; and transmitting the digitized covering state wirelessly or through a wired connection from the covering control device.
